(19) United States
(12) Patent Application Publication
(10) Pub. No.: US 2021/0301281 A1
(43) Pub. Date: Sep. 30, 2021

(54) IMMOBILIZED ENZYME FOR DIRECTIONALLY CATALYZING ESTERIFICATION REACTION AND PREPARATION METHOD AND USE THEREOF

(71) Applicant: Guangdong Pharmaceutical University, Zhongshan City (CN)

(72) Inventors: Nanjing ZHONG, Zhongshan City (CN); Wenyi CHEN, Zhongshan City (CN)

(21) Appl. No.: 17/139,245

(22) Filed: Dec. 31, 2020

(30) Foreign Application Priority Data
Mar. 27, 2020 (CN) 202010226830.1

(51) Int. Cl. C12N 11/02

(52) U.S. Cl. CPC C12N 11/02

(57) ABSTRACT

An immobilized enzyme for directionally catalyzing esterification reaction and a preparation method thereof are disclosed. The method includes the following steps: mixing a Candida antarctica lipase with a HEPES buffer solution to prepare a HEPES-lipase solution; mixing the HEPES-lipase solution with a nucleotide disodium salt mother liquor to obtain a mixed solution; adding a metal ion source and mixing the mixed solution to be uniform; then filtering and lyophilizing to obtain an immobilized enzyme.

### CROSS REFERENCE TO RELATED APPLICATION

The present application claims priority to Chinese Application No. 202010226830.1, filed on Mar. 27, 2020, entitled “Immobilized enzyme for directionally catalyzing esterification reaction and preparation method and use thereof,” which is hereby incorporated herein by reference in its entirety.

### FIELD OF THE INVENTION

The present disclosure relates to the technical field of biochemical industry, and particularly to an immobilized enzyme for directionally catalyzing esterification reaction and a preparation method and a use thereof.

### BACKGROUND OF THE INVENTION

Lipase (E.C3.1.1.3), i.e. triacylglyceride hydrolase, is used to catalyze the hydrolysis of natural oils to produce diglycerides, monoglycerides, free fatty acids and glycerol, and the thorough hydrolysis of the oils to produce free fatty acids and glycerol. Besides the hydrolysis reaction, lipase can be used to catalyze other reactions, including esterification reactions, transesterification reactions, and alcoholysis reactions. Due to these properties, the lipase can be widely used in food, chemical, pharmaceutical, and other industries. However, free lipase has many shortcomings, such as poor stability, the ability to be easily inactivated, inability to be recycled and reused, and difficulties in separation after being mixed into products. Therefore, there are great difficulties in the industrial application of lipase. The immobilization of lipase can realize its reuse Immobilized enzymes with excellent enzymatic properties (high enzyme activity, good stability, etc.) can be obtained by using suitable supports and immobilization technology.

For the immobilization of lipase, most of the current research focuses on improving the basic enzymatic properties such as enzyme activity, thermal stability, reusability, and pH tolerance. There are relatively few studies on the selective catalysis (directed catalysis) of enzymes. Lipase can not only catalyze hydrolysis reactions, but can also catalyze esterification reactions, transesterification reactions, alcoholysis reactions and other reactions. However, the fact that lipase can catalyze more reactions simultaneously can cause the undesirable formation of by-products in the actual process. For example, in the enzymatic deacidification process of high acid oils or in the esterification process for preparing a specific triglyceride, lipase is used to catalyze the esterification reaction to convert free fatty acids into triglycerides; however, because lipase can also catalyze glycerolysis reaction, which makes the (generated) triglycerides react with glycerol to generate monoglycerides and diglycerides, the content of the target product triglycerides is reduced. Therefore, the immobilized enzymes with directed catalytic properties have particularly important application prospects.

### SUMMARY OF THE INVENTION

In order to overcome the above shortcomings and deficiencies of the prior art, one objective of the embodiments of the present disclosure is to provide a method for preparing an immobilized enzyme for directionally catalyzing esterification reaction.

Another objective of the embodiments of the present disclosure is to provide an immobilized enzyme for directionally catalyzing esterification reaction as prepared by the above method.

Yet another objective of embodiments of the present disclosure is to provide a use of the above-mentioned immobilized enzyme for directionally catalyzing esterification reaction as a biological enzyme catalyst.

The objectives of the embodiments of the present disclosure could be achieved by the following technical solutions:

The present disclosure provides a method for preparing an immobilized enzyme for directionally catalyzing esterification reaction, including the following steps:

mixing a Candida antarctica lipase with a HEPES buffer solution, to prepare a HEPES-lipase solution; then mixing the HEPES-lipase solution with a nucleotide disodium salt mother liquor to obtain a mixed solution; then adding a metal ion source and mixing to be uniform; then filtering and lyophilizing to obtain an immobilized enzyme.

In some embodiments, the HEPES (N-2-hydroxyethylpiperazine-N′-2-ethanesulfonic acid) buffer solution has a concentration of 0.07-0.35 g/mL, and a pH value of 5.8-8.3;

In some embodiments, the concentration (protein concentrations) of the Candida antarctica lipase in the HEPES-lipase solution is in the range of 0.36-12.3 mg/mL.

In some embodiments, the nucleotide disodium salt is at least one selected from the group consisting of GMP (guanosine 5′-monophosphate disodium salt) and AMP (adenosine 5′-monophosphate disodium salt).

In some embodiments, the nucleotide disodium salt mother liquor has a concentration of 0.014-0.08 g/mL.

In some embodiments, a volume ratio of the HEPES-lipase solution to the nucleotide disodium salt mother liquor is in the range of 0.2-1:1. For example, the volume ratio of the HEPES-lipase solution to the nucleotide disodium salt mother liquor could be in the range of 0.4-0.6:1.

In some embodiments, the metal ion in the metal ion source is at least one selected from the group consisting of lanthanum (La³⁺), cerium (Ce³⁺), praseodymium (Pr³⁺), neodymium (Nd³⁺), samarium (Sm³⁺), europium (Eu³⁺), gadolinium (Gd³⁺), terbium (Tb³⁺), dysprosium (Dy³⁺), holmium (Ho³⁺), erbium (Er³⁺), thulium (Tm³⁺), ytterbium (Yb³⁺), lutetium (Lu³⁺), scandium (Sc³⁺), yttrium (Y³⁺), zinc (Zn²⁺), copper (Cu²⁺), iron (Fe³⁺), cadmium (Cd²⁺), and zirconium (Zr⁴⁺).

In some embodiments, the metal ion source is used in such an amount that a concentration of the metal ion in the mixed solution may be in the range of 0.03-0.31 mol/L.

Moreover, the present disclosure provides an immobilized enzyme for directionally catalyzing esterification reaction as prepared by the above method.

Furthermore, the present disclosure provides a use of the immobilized enzyme for directionally catalyzing esterification reaction as a biological enzyme catalyst.

The term “directionally,” as used in “immobilized enzyme for directionally catalyzing esterification reaction,” specifically refers to the ability to efficiently catalyze esterification reaction(s) but not catalyze alcoholysis reaction(s).

The alcoholysis reaction(s) includes: (1) the reaction of glycerol with triglycerides, (2) the reaction of monoglycerides with triglycerides, and (3) the reaction(s) of other fatty alcohols such as methanol, ethanol, and butanol with triglycerides.

Compared with the prior art, the embodiments of the present disclosure have the following advantages and beneficial effects:

The method for preparing the immobilized enzyme according to the present disclosure is simple; the immobilized enzyme prepared by the same has high enzymatic activity, can efficiently catalyze esterification reaction(s), and has good reusability. However, it cannot catalyze alcoholysis reaction(s); that is to say, the immobilized enzyme has directional catalytic properties. More particularly, it can catalyze esterification reaction(s), but cannot catalyze alcoholysis reaction(s). In view of this, the immobilized enzyme has very good application prospects.

### DETAILED DESCRIPTION OF THE EMBODIMENTS

The present disclosure will be further described in detail below in reference to examples, but the embodiments of the present disclosure are not limited thereto.

Unless otherwise specified, the reagents used in the examples can be commercially available. The Candida antarctica lipase B used in the examples was purchased from Novozyme, Beijing, China.

### EXAMPLE 1

(1) Preparation of a HEPES buffer solution: 2.0 g of HEPES was weighed and dissolved in water, and the resulting solution was adjusted to have a pH value of 6.5, diluted to a final volume of 25 mL, and stored at a low temperature.

(2) Preparation of a HEPES-lipase solution: 100 mg of Candida antarctica lipase B was weighed and dissolved in the buffer solution obtained in step (1), and stored at a low temperature.

(3) Preparation of an AMP mother liquor: 0.2 g of AMP was weighed, dissolved in water, diluted to a final volume of 10 mL, and stored at a low temperature.

(4) Preparation of a Cu²⁺ mother liquor: 0.16 g of anhydrous copper chloride was weighed, dissolved in water, diluted to a final volume of 10 mL, and stored at a low temperature.

(5) Preparation of an immobilized enzyme: 0.4 mL of the HEPES-lipase solution was mixed with 1.0 mL of the AMP mother liquor, then 1.2 mL of the Cu²⁺ mother liquor was added thereto and mixed. The resulting mixture was left standing, and centrifuged. After removing the supernatant, the filter cake was lyophilized to obtain the immobilized enzyme.

(6) The obtained immobilized enzyme was used to catalyze the esterification reaction of glycerol with oleic acid. The reaction was conducted under the following conditions: 0.4605 g of glycerol, 4.2369 g of oleic acid, and 0.20 g of the immobilized enzyme were placed in a three-necked reaction flask, and the resulting mixture were stirred in an oil bath at 70° C. for 24 hours (200 rpm). The obtained product after the reaction has the following composition, as detected by HPLC-ELSD: oleic acid 5.05%, oleic acid monoglyceride 0.83%, oleic acid diglyceride 6.08%, and oleic acid triglyceride 88.04%.

(7) The obtained immobilized enzyme was used to catalyze the alcoholysis reaction of glycerol with soybean oil. The reaction was conducted under the following conditions: 0.184 g of glycerol, 3.52 g of soybean oil, and 0.15 g of the immobilized enzyme were placed in a single-neck reaction flask, and the resulting mixture was stirred in an oil bath at 60° C. for 12 hours (200 rpm). The composition of the product after the reaction was detected by HPLC-ELSD, and the results showed that the conversion rate for soybean oil was almost zero; that is to say, the glycerolysis reaction was not performed.

### EXAMPLE 2

(1) Preparation of a HEPES buffer solution: 3.0 g of HEPES was weighed and dissolved in water, and the resulting solution was adjusted to have a pH value of 6.5, diluted to a final volume of 40 mL, and stored at a low temperature.

(2) Preparation of a HEPES-lipase solution: 200 mg of Candida antarctica lipase B was weighed and dissolved in the buffer solution obtained in step (1), and stored at a low temperature.

(3) Preparation of a GMP mother liquor: 0.3 g of GMP was weighed, dissolved in water, diluted to a final volume of 10 mL, and stored at a low temperature.

(4) Preparation of a Tb³⁺ mother liquor: 0.37 g of terbium chloride hexahydrate was weighed, dissolved in water, diluted to a final volume of 10 mL, and stored at a low temperature.

(5) Preparation of an immobilized enzyme: 0.6 mL of the HEPES-lipase solution was mixed with 1.0 mL of the GMP mother liquor, then 1.0 mL of the Tb³⁺ mother liquor was added thereto and mixed. The resulting mixture was left standing, and centrifuged. After removing the supernatant, the filter cake was lyophilized to obtain the immobilized enzyme.

(6) The obtained immobilized enzyme was used to catalyze the esterification reaction of glycerol with palmitic acid. The reaction was conducted under the following conditions: 0.4605 g of glycerol, 3.846 g of palmitic acid, and 0.20 g of the immobilized enzyme were placed in a three-necked reaction flask, and the resulting mixture were stirred in an oil bath at 70° C. for 24 hours (200 rpm). The obtained product after the reaction has the following composition, as detected by HPLC-ELSD: palmitic acid 0.9%, palmitic acid monoglyceride 0.63%, palmitic acid diglyceride 5.14%, and palmitic acid triglyceride 93.33%.

(7) The obtained immobilized enzyme was used to catalyze the alcoholysis reaction of glycerol with palm oil, and the reaction was conducted under the following conditions: 0.184 g of glycerol, 3.20 g of palm oil, and 0.20 g of the immobilized enzyme were placed in a single-neck reaction flask, and the resulting mixture was stirred in an oil bath at 60° C. for 12 hours (200 rpm). The composition of the product after the reaction was detected by HPLC-ELSD, and the results showed that the conversion rate for palm oil was almost zero; that is to say, the glycerolysis reaction was not performed.

### EXAMPLE 3

(1) Preparation of a HEPES buffer solution: 5.0 g of HEPES was weighed and dissolved in water, and the resulting solution was adjusted to have a pH value of 8.0, diluted to a final volume of 50 mL, and stored at a low temperature.

(2) Preparation of a HEPES-lipase solution: 300 mg of Candida antarctica lipase A was weighed and dissolved in the buffer solution obtained in step (1), and stored at a low temperature.

(3) Preparation of a GMP mother liquor: 0.3 g of GMP was weighed, dissolved in water, diluted to a final volume of 10 mL, and stored at a low temperature.

(4) Preparation of a Zn²⁺ mother liquor: 0.15 g of anhydrous zinc chloride was weighed, dissolved in water, diluted to a final volume of 10 mL, and stored at a low temperature.

(5) Preparation of an immobilized enzyme: 0.5 mL of the HEPES-lipase solution was mixed with 1.0 mL of the GMP mother liquor, then 1.2 mL of the Zn²⁺ mother liquor was added thereto and mixed. The resulting mixture was left standing, and centrifuged. After removing the supernatant, the filter cake was lyophilized to obtain the immobilized enzyme.

(6) The obtained immobilized enzyme was used to catalyze the esterification reaction of glycerol and oleic acid. The reaction was conducted under the following conditions: 0.4605 g of glycerol, 3.0045 g of lauric acid, and 0.18 g of the immobilized enzyme were placed in a three-necked reaction flask, and the resulting mixture were stirred in an oil bath at 70° C. for 24 hours (200 rpm). The obtained product after the reaction has the following composition, as detected by HPLC-ELSD: lauric acid 4.65%, lauric acid monoglyceride 0.92%, lauric acid diglyceride 7.21%, and lauric acid triglyceride 87.22%.

(7) The obtained immobilized enzyme was used to catalyze the alcoholysis reaction of glycerol with corn oil. The reaction was conducted under the following conditions: 0.184 g of glycerol, 3.52 g of corn oil, and 0.20 g of the immobilized enzyme were placed in a single-neck reaction flask, and the resulting mixture was stirred in an oil at 60° C. bath for 12 hours (200 rpm). The composition of the product after the reaction was detected by HPLC-ELSD, and the result showed that the conversion rate for corn oil was almost zero; that is to say, the glycerolysis reaction was not performed.

The above embodiments are preferred embodiments of the present disclosure, but the embodiments of the present disclosure are not limited by the same. Any other variations, modifications, substitutions, combinations, or simplifications in the specifically-described embodiments may be carried out without departing from the spirit and principle of the present disclosure and its equivalent replacements, and should fall within the scope of the present disclosure, which is intended to be limited only by the scope of the appended claims as interpreted according to the principles of patent law including the doctrine of equivalents.

1. A method for preparing an immobilized enzyme for directionally catalyzing esterification reaction, comprising:
mixing a Candida antarctica lipase with a HEPES buffer solution to prepare a HEPES-lipase solution;
mixing the HEPES-lipase solution with a nucleotide disodium salt mother liquor to obtain a mixed solution;
adding a metal ion source to the mixed solution and mixing the mixed solution to be uniform; and
filtering and lyophilizing the mixed solution to obtain an immobilized enzyme.

2. The method for preparing an immobilized enzyme for directionally catalyzing esterification reaction as claimed in claim 1, wherein the concentration of the Candida antarctica lipase in the HEPES-lipase solution is in the range of 0.36-12.3 mg/mL.

3. The method for preparing an immobilized enzyme for directionally catalyzing esterification reaction as claimed in claim 1, wherein the HEPES buffer solution has a concentration of 0.07-035 g/mL, and a pH value of 5.8-8.3.

4. The method for preparing an immobilized enzyme for directionally catalyzing esterification reaction as claimed in claim 1, wherein the nucleotide disodium salt is at least one selected from the group consisting of guanosine 5′-monophosphate disodium salt and adenosine 5′-monophosphate disodium salt.

5. The method for preparing an immobilized enzyme for directionally catalyzing esterification reaction as claimed in claim 1, wherein a volume ratio of the HEPES-lipase solution to the nucleotide disodium salt mother liquor is in the range of 0.2-1:1.

6. The method for preparing an immobilized enzyme for directionally catalyzing esterification reaction as claimed in claim 1, wherein a metal ion in the metal ion source is at least one selected from the group consisting of La³⁺, Ce³⁺, Pr³⁺, Nd³⁺, Sm³⁺, Eu³⁺, Gd³⁺, Tb³⁺, Dy³⁺, Ho³⁺, Er³⁺, Tm³⁺, Yb³⁺, Lu³⁺, Sc³⁺, Y³⁺, Zn²⁺, Cu²⁺, Fe³⁺, Cd²⁺, and Zr⁴⁺.

7. The method for preparing an immobilized enzyme for directionally catalyzing esterification reaction as claimed in claim 1, wherein the metal ion source is used in such an amount that a concentration of the metal ion in the mixed solution is in the range of 0.03-0.31 mol/L.

8. An immobilized enzyme for directionally catalyzing esterification reaction, as prepared by the method as claimed in claim 1.

9. The method for preparing an immobilized enzyme for directionally catalyzing esterification reaction as claimed in claim 1, wherein the nucleotide disodium salt mother liquor has a concentration of 0.014-0.08 g/mL.
